(19) United States
(12) Patent Application Publication
(10) Pub. No.: US 2015/0160199 A1
(43) Pub. Date: Jun. 11, 2015

(54) DIAGNOSIS DEVICE HAVING LINEAR TRANSPORT FUNCTION AND APPLICATION THEREOF

(71) Applicant: National Tsing Hua University, Hsinchu (TW)

(72) Inventors: HSI-KAI WANG, HSINCHU (TW); KUAN-HUNG CHEN, HSINCHU (TW); CHEN-MENG KUAN, HSINCHU (TW); CHAO-MIN CHENG, HSINCHU (TW)

(73) Assignee: National Tsing Hua University, Hsinchu (TW)

(21) Appl. No.: 14/159,782

(22) Filed: Jan. 21, 2014

(30) Foreign Application Priority Data
Dec. 6, 2013 (TW) 102144835

(51) Int. Cl. G01N 33/52; B01L 3/00

(52) U.S. Cl. CPC G01N 33/528; B01L 3/5023; B01L 2300/161; B01L 2300/0887; B01L 2300/089; B01L 2400/0406

(57) ABSTRACT

A diagnosis device having linear transport function with capillarity action, which includes a test paper and a linear transporting means. The present invention is configured with a linear transporting means so as to achieve clear colorimetric effects without being interfered by original liquid color during diagnosis. In addition, the present invention may achieve controlled colorimetric region by confined hydrophilic area defined by hydrophobic area. Therefore, the present invention has advantages in being fast, easy to observe and cheap. A flushing bag with a linear transporting means having diagnostic capability is also herein provided, wherein the other end of the linear transporting means is connected to the flushing bag such as a tea bag, a hot spring bag or an herb medicine bag. The linear transporting means may be a cotton thread or a strip or other cellulose-based/non-cellulose-based material with capillarity transporting function.

### BACKGROUND OF THE INVENTION

1. Field of the Invention

The present invention relates to a diagnosis device, and particularly relates to a diagnosis device having linear transport function.

2. Description of the Prior Art

With the increasing trend in people's health awareness, the concept of self-diagnosis/self-testing at home has gradually prevailed. Self-test kits provide tests that people can perform tests at home at any time and may be achieved by observing the color change of the detection reagent. Therefore, disease signs can be immediately observed with simple instruments or unaided eyes instead of complex instruments. In addition, when the chromatic aberration grows massive, users may go to the hospital for further detailed diagnosis. Therefore, self-test kits have advantages in immediate facilitation and saving money.

Regarding self-test kits used at home, test strips have been frequently used. Test strips are provided with advantages such as simple operation, easy to interpret and easy to carry. The principle of test strip detection is achieved by biochemical reactions catalyzed by the enzyme and specific substances to generate changes in color or other properties, and a qualitative reaction for detection the presence of a substance or a semi-quantitative reaction for determining the concentration of the substance may thus be achieved.

For example, a glucose oxidase method applied in home-use blood sugar or urine glucose test strips refers to the color changes in test strip achieved by the specificity of the catalytic reaction between glucose oxidase and glucose while other reducing substances does not work. A test strip coated with a glucose oxidase using enzyme technology would result in color changes after the reaction of glucose oxidase and glucose in the blood, so that the user can interpret glucose concentration level based on comparison resulting color of the test strip with the color chart.

This simple blood glucose measurement method has been found beneficial to diabetics, such as monitoring disease at any time in terms of improved quality of life as well as expanded scope of medical care. However, the convenience of the test strip is still room for improvement since the test strip is still in need of special carry. Therefore, the development of a testing platform, which has the advantages of the test strip and has the convenience of matching with the daily necessities, is a current goal.

### SUMMARY OF THE INVENTION

The present invention is directed to providing a detection platform, which has advantages of test strip and is compatible to daily necessities.

According to one embodiment of the present invention, a diagnosis device having linear transport function comprises a test strip, at least one detection reagent and a linear transporting means. The test strip comprises a hydrophobic area and a hydrophilic area, wherein the hydrophilic area is substantially surrounded by the hydrophobic area. The detection reagent is coated on the hydrophilic area. The linear transporting means is connected to the hydrophilic area of the test strip, whereby a liquid specimen or a specimen containing liquid is in contact with the linear transporting means, the specimen is adsorbed to the linear transporting means by capillary action to reach the hydrophilic area of the test strip to react with the detection reagent so as to achieve the role of specimen detection.

According to another embodiment of the present invention, a linear transport diagnosis device comprises a test strip, at least one detection reagent, a flushing bag and a linear transporting means. The test strip comprises a hydrophobic area and a hydrophilic area, wherein the hydrophilic area is substantially surrounded by the hydrophobic area. The detection reagent is coated on the hydrophilic area. The linear transporting means is connected to the flushing bag and the hydrophilic area of the test strip, whereby the flushing bag is immersed in a liquid to obtain a liquid specimen, the liquid specimen is absorbed with capillarity to the flushing bag and the linear transporting means and transmitted to the hydrophilic area of the test strip to react with the detection reagent for detecting the liquid specimen.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

FIG. 1 is a schematic view showing a diagnosis device having linear transport function of the present invention.

FIGS. 2 to 4 are data showing the effect of the diagnosis device having linear transport function of the present invention.

### DESCRIPTION OF THE PREFERRED EMBODIMENT

The present invention relates to a diagnosis device having linear transport function includes a test strip 1, and a linear transporting means 2. The test strip 1 has a hydrophobic area 12 and a hydrophilic area 11, wherein the hydrophilic area 11 is substantially surrounded by the hydrophobic area 12. At least one detection reagent is coated onto the hydrophilic area 11. The hydrophilic area 11 defined by the hydrophobic area 12 may limit the range of the aqueous solution of the test strip 1 so as to control the size range colorimetric area for easier observation and prevent the test strip 1 from being too wet and increase utility.

The size, number and shape of the hydrophilic area 11 of the test strip 1 are not particularly limited. The hydrophilic area 11 may be made of celluloses or synthetic polymers, and be porous and absorbent, preferably. In one preferred embodiment, the hydrophilic area 11 may be made of the materials including without being limited to filter paper or nitrocellulose membranes. The chromatography filter paper of the present invention is semi-permeable, which may be used for phase separation. For example. It can separate solid substance from liquid or air.

Materials of filter papers are plant fibers which are usually derived from lumbers and cottons. In one preferred embodiment, the chromatography filter papers are Whatman® cellulose chromatography filter papers made of cotton fibers, which are commercially available.

People skilled in the art should understand different ways for defining the hydrophobic region 12 of the chromatography filter paper plate 1. For instance, in one preferred embodiment of the present invention, the hydrophobic area 12 is formed by coating chemical materials on the paper plate such as wax printing.

In one embodiment, specific patterns can be printed on the chromatography filter paper by wax printing. After that, the patterned chromatography filter paper is baked (100□, 10 min) to obtain the chromatography filter paper plate 1 of the present invention.

People skilled in the art should understand there are different methods for preparing the chromatography filter paper plate 1. In one embodiment, after coating a photoresist layer SU-8 thereon and followed by UV irradiation, the hydrophobic area 12 is formed to define the hydrophilic regions 11.

Further, in one embodiment, the hydrophilic area 11 and hydrophobic area 12 may be set in a three-dimensional manner, i.e., the hydrophilic area 11 may be surrounded by upper and lower hydrophobic areas 12.

The diagnosis device having linear transport function of the present invention is provided with a linear transporting means 2, which is connected to the hydrophilic area 11 of the test strip 1. Here, when a liquid specimen or a specimen containing liquid is in contact with the linear transporting means 2, the specimen is adsorbed to the linear transporting means 2 by capillary action to reach the hydrophilic area 11 of the test strip 1 to react with the detection reagent so as to achieve the role of specimen detection. The considerations regarding the materials of the linear transporting means 2 are mainly based on absorbability. In other words, so long as a good absorbent material is applicable in principle to applying capillary force to siphon liquid test specimen for transportation. The linear transporting means 2 may be a cotton thread or a strip or other cellulose-based/non-cellulose-based material with capillarity transporting function. The linear transporting means 2 is preferably made of cotton fiber and the linear transporting means may be a linear line or curve. The width and the length of the linear transporting means 2 are not particularly limited.

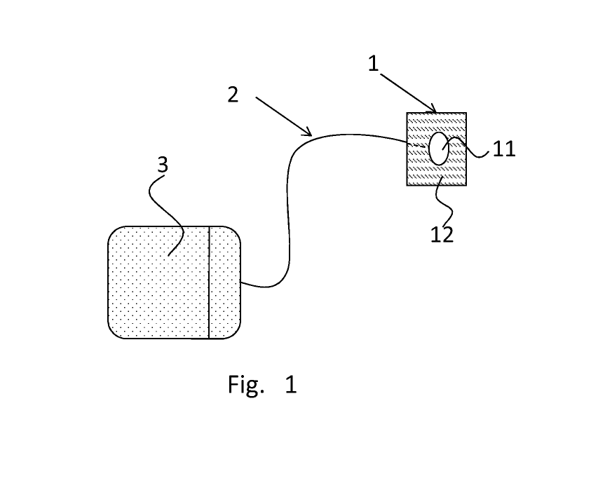

Still referring to FIG. 1, in another embodiment, the diagnosis device having linear transport function of the present invention is a flushing bag 3 with diagnostic function, wherein the hydrophilic area 11 of the test strip 1 and the flushing bag 3 are connected with the linear transporting means 2. When the flushing bag 3 is immersed in a liquid to obtain a liquid specimen, the liquid specimen is absorbed with capillarity to the flushing bag 3 and the linear transporting means 2 and transmitted to the hydrophilic area 11 of the test strip 1 to react with the detection reagent for detecting the liquid specimen. The size of the flushing bag 3 is not particularly limited. Thus, the advantages of the present invention include clear colorimetric effects that are not affected by the original color of the liquid specimen during detection as well and being fast, simple and inexpensive.

Those skilled in the art should understand many ways to connect the linear transporting means 2 to the flushing bag 3 and the hydrophilic area 11 of the test strip 1, for example without being limited to adhesives or glues. Otherwise, the linear transporting means 2 may penetrate the flushing bag 3 or the test strip 1 to achieve the connection.

It is stated herein that the “flushing bag” refers to packed device for flushing and may be flushed with liquid (such as water) to become drinks or other function. The flushing bag may include, but are not limited to a tea bag, a hot spring bag or an herb medicine bag. Other examples such as coffee bag, flower tea bag are also within the scope of the present invention.

In addition, in one embodiment, the test strip 1 may be used as a tag of the flushing bag 3.

The test strip 1 for detection of the present invention may be detected by colorimetric reaction or fluorescence, accordingly the detection reagent comprises a colorimetric agent or a fluorescent reagent. Colorimetric agents or fluorescent reagents used as detection reagents may be chosen based on various subject matters targets to be detected and make adaptive changes.

In one embodiment of the present invention, the detection reagent may comprise an enzyme. As above-mentioned, qualitative measurements for detecting the presence of substances and quantitative measurements for determining the substance concentration may be performed by measuring changes in color or other properties caused by reaction between enzyme and substances in the solution. For example, color changes of the test strip 1 using the glucose oxidase assay is achieved by the specific reaction catalyzed by glucose oxidase and glucose.

Examples of the specimen include without limitations to a subject's saliva, blood, urine or other body fluids. As mentioned above, the specimen may be absorbed to the sample test area of the test strip 1 by capillary action to react with the detection reagent to achieve specimen detection. In addition, solid substances to be detected may be suspended and dissolved in a liquid solvent and then be detected by the above-described detection method using capillary action.

Continuing the above description, the test strip for detection of the present invention may be used for testing biochemical properties, such as glucose, nitrite, pH, of saliva, blood, urine or other body fluids. The following examples are only used for illustrating the principle and applications of detection.

### Example 1

### Nitrite Detection

In the case of nitrate ions present in the food, nitrosamines which have been identified as a carcinogen by the medical researchers are formed by action of digestive enzymes in the saliva. In addition, large amount of nitrite may cause direct poisoning after uptaking nitrite-abundant water, vegetables, grain, fish, meat, salted products. At present, there are commercially available nitrite test strips using chemical colorimetric reaction; therefore, nitrite detection in food would be a suitable target for detection.

### Example 2

### Glucose Detection

The measurement principle for blood sugar or urine glucose has been described above. Patients can use glucose detection device so as to monitor their conditions at any time.

### Example 3

### PH Detection

Regarding the detection of pH, Litmus paper is commonly used to measure the pH of the solution. Litmus paper is made of paper immersed in the solution containing litmus reagent. The litmus reagent would indicate red in the acidic solution, and blue in the alkaline solution. The test strip may be prepared using the aforementioned properties so as to detect the pH value.

The present invention is further illustrated by the following working examples, which should not be construed as further limiting.

### Example 4

### Comparison of the Design of the Detection Zone of the Test Strip

The design pattern (the hydrophobic area and the hydrophilic area) of test strip was achieved by using a wax printing method and a hydrophobic restricted area was created by wax printing properties to form the diagnosis device and limit the hydrophilic area of the test strip to control the colorimetric range and size.

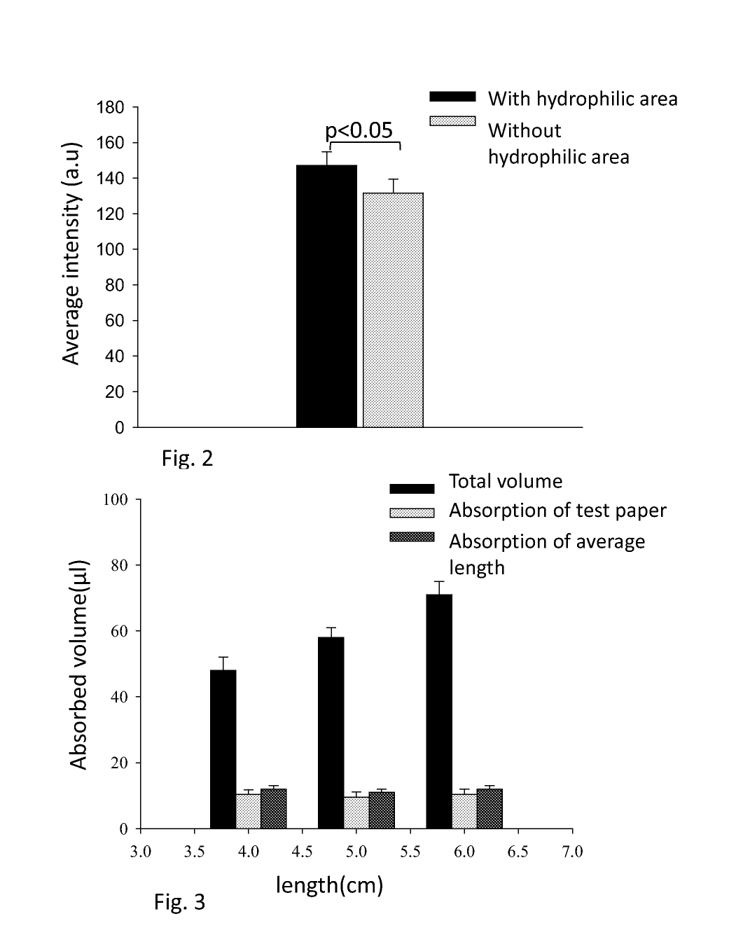

Referring to FIG. 2, test strips with and without design patterns used as sample detection area were compared. When blue water passes through the linear transporting means to detect two different test strips, comparison for colorimetric intensity is performed by use of the naked eye, a camera or a scanner to collect images. The results showed that the dark group with a specific printing pattern has statistically significant difference (p<0.05) from light gray group of the test strip without the printing pattern.

### Example 5

### Testing of Transportation Volume of the Linear Transporting Means

A specific linear transporting means with a fixed length was selected, a precision weighing scale (sensitivity of 1 mg) was used to measure the weight of the linear transporting means that was not absorbent with water. The linear transporting means was hanged at the rim of the beaker with only 1 to 2 cm end in touch with the water. The other end of the linear transporting means was then absorbent with solution by capillarity and the weight of the linear transporting means was measured again. The linear transporting means was put to the upper surface of the test strip to form a diagnosis device. Due to the stronger absorbability of the test strip, the solution contained in the linear transporting means transferred to the test strip until the detection area was fully absorbent with water. The weight of the linear transporting means was measured again. Processes were within 3 minutes; therefore, the water evaporation can be ignored in comparison to the amount of water absorption. All experiments were performed at room temperature, and the density of water can be regarded as 1 g/cm³, in terms of 1000 mg/mL, i.e., transfer of 1 mg equals to 1 μL water.

In the present invention, the linear transporting means may be used for replacing the conventional quantitative pipette to deliver detected sample to the sample detection area. Referring to FIG. 3, the absorbed volume of the sample of the linear transporting means is proportional to its length; this means that the absorbed volume of the sample per centimeter is fairly consistent. It is found from the experiment that the volume transferred from the linear transporting means to saturate the detection area is not affected by the length of the linear transporting means and the saturated volume transferred to the detection area is fixed to have a stable reproducibility.

### Example 6

### Nitrite Test

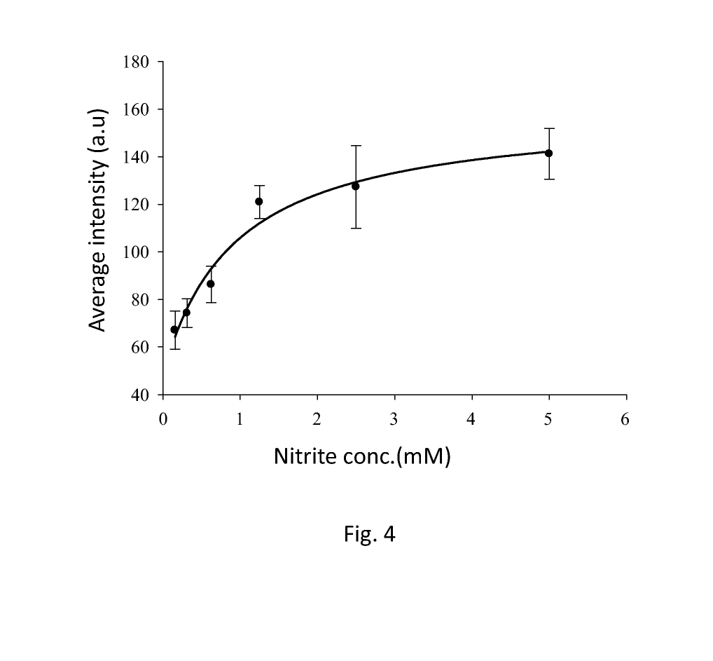

4 μL of nitrite detection reagent was dipped on the detection zone of the device with quantitative pipette till the solution gradually dried. An end of the linear transporting means was immersed into nitrite solution at different concentrations and the detection means was hanged at the rim of the cup. The test strip was hanged outside the plate without contact to the solution. The entire device was hanged on the back into a U-shape. Nitrite solution is reached via the linear transporting means to the detection zone on the test strip to perform nitrite color reaction. Referring to FIG. 4, the diagnosis device of the present invention can be used to detect nitrite. Therefore, the present invention has advantages in being fast, easy to observe and cheap.

In summary, the present invention is configured with a linear transporting means so as to achieve clear colorimetric effects without being interfered by original liquid color during diagnosis. In addition, the present invention may achieve controlled colorimetric region by confined hydrophilic area defined by hydrophobic area and therefore achieve advantages such as easy observation and rapid detection.

In addition, the invention may be used for replacing conventional detection that requires special operation of the appliance/well-trained professionals and collecting samples to be tested so as to achieve advantage of reducing testing costs.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A diagnosis device having linear transport function, comprising:
a test strip comprising a hydrophobic area and a hydrophilic area, wherein the hydrophilic area is substantially surrounded by the hydrophobic area;
at least one detection reagent coated on the hydrophilic area; and
a linear transporting means connected to the hydrophilic area of the test strip, whereby a liquid specimen or a specimen containing liquid is in contact with the linear transporting means, the specimen is adsorbed to the linear transporting means by capillary action to reach the hydrophilic area of the test strip to react with the detection reagent so as to achieve the role of specimen detection.

2. The diagnosis device as claimed in claim 1, wherein the hydrophilic area of the test strip is mainly made of cotton fiber.

3. The diagnosis device as claimed in claim 1, wherein the hydrophobic area is formed by wax printing.

4. The diagnosis device as claimed in claim 1, wherein the linear transporting means is made of cotton fiber.

5. The diagnosis device as claimed in claim 1, wherein the detection reagent comprises a colorimetric agent or a fluorescent reagent.

6. The diagnosis device as claimed in claim 1, wherein the detection reagent comprises an enzyme.

7. The diagnosis device as claimed in claim 1, wherein the fluid specimen is saliva, blood or urine from a subject.

8. The diagnosis device as claimed in claim 1, being a glucose, nitrite, or pH detection device.

9. A linear transport diagnosis device, comprising:
a test strip comprising a hydrophobic area and a hydrophilic area, wherein the hydrophilic area is substantially surrounded by the hydrophobic area;
at least one detection reagent coated on the hydrophilic area;
a flushing bag; and
a linear transporting means connected to the flushing bag and the hydrophilic area of the test strip, whereby the flushing bag is immersed in a liquid to obtain a liquid specimen, the liquid specimen is absorbed with capillarity to the flushing bag and the linear transporting means and transmitted to the hydrophilic area of the test strip to react with the detection reagent for detecting the liquid specimens.

10. The diagnosis device as claimed in claim 9, wherein the flushing bag includes a tea bag, a hot spring bag or an herb medicine bag.

11. The diagnosis device as claimed in claim 9, wherein the hydrophilic area of the test strip is mainly made of cotton fiber.

12. The diagnosis device as claimed in claim 9, wherein the hydrophobic area is formed by wax printing.

13. The diagnosis device as claimed in claim 9, wherein the linear transporting means is made of cotton fiber.

14. The diagnosis device as claimed in claim 9, wherein the detection reagent comprises a colorimetric agent or a fluorescent reagent.

15. The diagnosis device as claimed in claim 9, wherein the detection reagent comprises an enzyme.

16. The diagnosis device as claimed in claim 9, wherein the fluid specimen is saliva, blood or urine from a subject.

17. The diagnosis device as claimed in claim 9, being a glucose, nitrite, or pH detection device.
